(19) United States
(12) Patent Application Publication
(10) Pub. No.: US 2023/0250413 A1
(43) Pub. Date: Aug. 10, 2023

(54) ENZYME-IMMOBILIZATION CARRIER AND IMMOBILIZED ENZYME THAT USES SAME

(71) Applicant: FUJI OIL HOLDINGS INC., Osaka (JP)

(72) Inventor: Shigeki MIZUSHIMA, Ibaraki (JP)

(73) Assignee: FUJI OIL HOLDINGS INC., Osaka (JP)

(21) Appl. No.: 18/012,979

(22) PCT Filed: Oct. 5, 2021
(86) PCT No.: PCT/JP2021/036729

§ 371 Date: Dec. 27, 2022

(30) Foreign Application Priority Data
Oct. 19, 2020 (JP) 2020-175092

(51) Int. Cl. C12N 11/02; C12P 7/64; C12N 9/20; A23D 9/007; A23D 9/04; A23L 29/00

(52) U.S. Cl. CPC C12N 11/02; A23D 9/04; A23D 9/007; A23L 29/06; C12N 9/20; C12P 7/64

(57) ABSTRACT

The present invention addresses the objective of providing a novel, reproducible plant-based enzyme-immobilization carrier that is biodegradable and imposes a low environmental load and is also harmless for the human body. The technical concept of the present invention was achieved with the discovery, in response to the objective, that a prescribed porous body based on plant protein, e.g., from soy, functions as an enzyme-immobilization carrier.

### TECHNICAL FIELD

The present invention relates to a carrier used for immobilizing an enzyme, and an immobilized enzyme using the same.

### BACKGROUND ART

Modification of fats and oils is widely performed by a transesterification reaction of fats and oils using lipase as a catalyst. In the transesterification reaction of these fats and oils, immobilized enzymes, in which lipase is immobilized on some kind of carrier, have conventionally been used as catalysts in many cases.

As examples of carriers used for immobilization of enzymes, such as lipase, for fats and oils as a substrate, anion exchange resins (Patent Literature 1 and Patent Literature 3), phenol formaldehyde adsorption resins (Patent Literature 2), and chelate resins (Patent Literature 4) have been disclosed. In addition, Patent Literature 5 discloses that activated alumina, calcium carbonate, magnesium carbonate, magnesium oxide, diatomaceous earth, and the like are used as carriers that immobilize enzymes.

However, all of these carriers had low biodegradability. In addition, although diatomaceous earth is an inexpensive material and thus has been widely used as a carrier, resources are being depleted worldwide, and there has been a concern about future cost increases.

### CITATION LIST

### Patent Literature

[Patent Literature 1] Japanese Patent Laid-Open No. S60-98984
[Patent Literature 2] Japanese Patent Laid-Open No. S61-202688
[Patent Literature 3] Japanese Patent Laid-Open No. H3-61485
[Patent Literature 4] Japanese Patent Laid-Open No. H1-262795
[Patent Literature 5] Japanese Patent Laid-Open No. H11-69974
[Patent Literature 6] PCT International Publication No. WO2014/156948
[Patent Literature 7] Japanese Patent Laid-Open No. S47-3717

### SUMMARY OF INVENTION

### Technical Problem

An objective of the present invention is to provide a novel and reproducible plant-based enzyme-immobilization carrier that is biodegradable, thereby imposing a low environmental load, and is also harmless for a human body.

### Solution to Problem

As a result of intensive research on the above-mentioned objective, the inventor of the present invention found that a specific porous body based on plant protein such as soybean functions as an enzyme-immobilization carrier, whereupon the technical concept of the present invention was completed. An enzyme-immobilization carrier of the present invention is an invention relating to a product for which use application is limited because although the existence of a specific porous body itself including plant protein is known, this specific porous body is used for a conventionally unknown use application, that is, a carrier that immobilizes enzymes.

That is, the present invention relates to the following aspects.

An enzyme-immobilization carrier which has all of the following characteristics A to D:
A. the enzyme-immobilization carrier is a porous body including plant protein,
B. a content of the protein is 80% by weight or more per dry weight,
C. a nitrogen solubility index (NSI) is 70 or less, and
D. an oil absorption ratio is 2 times or more by weight.

The enzyme-immobilization carrier according to (1), in which the enzyme-immobilization carrier further has both of the following characteristics E and F:
E. a water absorption ratio is 7 times or more by weight, and
F. a bulk specific gravity is 0.28 g/cm³ or less.

An immobilized enzyme, in which the enzyme is immobilized on the enzyme-immobilization carrier according to (1) or (2).

The immobilized enzyme according to (3), in which the enzyme is for fats and oils as a substrate.

A method for producing an immobilized enzyme, the method including: adhering an aqueous solution containing an enzyme for fats and oils as a substrate to an enzyme-immobilization carrier having all of the following characteristics A to D; and drying thereafter.
A. the enzyme-immobilization carrier is a porous body including plant protein,
B. a content of the protein is 80% by weight or more per dry weight,
C. a nitrogen solubility index (NSI) is 70 or less, and
D. an oil absorption ratio is 2 times or more by weight.

A method for immobilizing an enzyme, the method including: adhering an aqueous solution containing an enzyme for fats and oils as a substrate to an enzyme-immobilization carrier having all of the following characteristics A to D; and drying thereafter.
A. the enzyme-immobilization carrier is a porous body including plant protein,
B. a content of the protein is 80% by weight or more per dry weight,
C. a nitrogen solubility index (NSI) is 70 or less, and
D. an oil absorption ratio is 2 times or more by weight.

A method for producing transesterified fats or oils, the method including performing a transesterification reaction using an immobilized enzyme obtained by adhering an aqueous solution containing an enzyme for fats and oils as a substrate to an enzyme-immobilization carrier having all of the following characteristics A to D, and drying thereafter.
A. the enzyme-immobilization carrier is a porous body including plant protein,
B. a content of the protein is 80% by weight or more per dry weight,
C. a nitrogen solubility index (NSI) is 70 or less, and
D. an oil absorption ratio is 2 times or more by weight.

### Advantageous Effects of Invention

According to the present invention, a specific porous body, which includes plant protein and is easily and continuously available, can be used as a carrier that immobilizes enzymes, thereby a novel immobilized enzyme can be obtained.

### Description of Embodiments

Hereinbelow, the present invention will be specifically described.

### Immobilization of Enzyme

An enzyme-immobilization carrier of the present invention is a carrier in a case where an enzyme is immobilized to be used in an enzymatic reaction. For the immobilization herein, there are physical and chemical methods, but an immobilization method is not limited in the present invention. However, since the present invention relates to an immobilized enzyme, immobilization methods that would deactivate enzymes are unusable. From this viewpoint, a physical method is preferable because it is an immobilization method that causes little damage to an enzyme protein.

### Enzyme

An immobilized enzyme of the present invention is preferably an immobilized enzyme for fats and oils as a substrate. This is because when a reaction is caused in an aqueous system, an enzyme immobilized on a carrier may be detached since enzymes are generally water-soluble, which may result in a deterioration in the reaction. The phrase “for fats and oils as a substrate” means that a reaction is caused in an oil system.

Although there is no limitation, lipases and phospholipases are desirable as the enzyme for fats and oils as a substrate.

Sometimes enzymes are traded as immobilized enzymes immobilized on some kind of carrier, and sometimes users cause immobilization by themselves after purchasing powder or liquid enzyme preparations. Implementation of the present invention includes both cases.

Hereinbelow, the characteristics of the enzyme-immobilization carrier of the present invention will be specifically described.

### Porous Body Based on Plant Protein

The enzyme-immobilization carrier of the present invention is a specific porous body including plant protein.

The plant protein is a protein acquired from a plant raw material, and examples thereof include proteins acquired from beans such as soybeans, peas, mung beans, chickpeas, peanuts, almonds, lupine, pigeon peas, sword beans, wild soybeans, common beans, adzuki beans, black-eyed beans, lentils, broad beans, and locust beans, seeds such as rapeseed (particularly canola varieties), sunflower seeds, cotton seeds, and coconuts, and cereals such as wheat, barley, rye, rice, and corn. Immobilized enzymes are often used in the processing of various food materials, but when plant proteins derived from the above-mentioned raw materials are used as raw materials, there is also the benefit of feeling relief when used because people have experience eating many types of plant proteins.

### Protein Content

The enzyme-immobilization carrier of the present invention is characterized by containing 80% by weight or more of protein per dry weight. The protein content may be 85% by weight or more, or 90% by weight or more. In addition, the content may be 99% by weight or less, 95% by weight or less, 90% by weight or less, 85% by weight or less, or 80% by weight or less per dry weight.

The protein content is obtained by multiplying a nitrogen amount analyzed by the Kjeldahl method by a nitrogen conversion factor of 6.25.

### Low Water Solubility

The enzyme-immobilization carrier of the present invention exhibits low water solubility. A Nitrogen Solubility Index (NSI) can be used as a water solubility index, and the lower the NSI, the lower the water solubility. The enzyme-immobilization carrier of the present invention has an NSI of 70 or less, preferably 50 or less, and more preferably 30 or less.

### Nitrogen Solubility Index (NSI)

The NSI of the enzyme-immobilization carrier of the present invention is preferably low. It is presumed that the reason for this is because when adhering an aqueous enzyme solution, an enzyme is prevented from being encompassed by protein, and the enzyme is distributed on the surface of the carrier.

An NSI can be expressed as a proportion (% by weight) accounting for water-soluble nitrogen (crude protein) in a total nitrogen amount based on a predetermined method, and is a value measured according to the following method in the present invention.

That is, 60 ml of water is added to 3 g of a sample, stirred with a propeller at 37° C. for 1 hour, and thereafter centrifuged at 1,400 x g for 10 minutes to collect a supernatant solution (1). Subsequently, 100 ml of water is added again to the remaining precipitate, and the mixture is stirred again with a propeller at 37° C. for 1 hour and thereafter centrifuged to collect a supernatant solution (2). The solution (1) and the solution (2) are combined, and water is added to the mixed solution so that a volume is 250 ml. After filtering this with filter paper (No. 5), the nitrogen content in the filtrate is measured by the Kjeldahl method. At the same time, the nitrogen amount in the sample is measured by the Kjeldahl method, and a ratio of the nitrogen amount (water-soluble nitrogen) recovered as a filtrate to the total nitrogen amount in the sample is expressed as % by weight, which is defined as an NSI.

### Bulk Specific Gravity

The bulk specific gravity of the enzyme-immobilization carrier of the present invention is preferably low and is specifically 0.28 g/cm³ or less, and more preferably 0.23 g/cm³ or less.

Generally, the bulk specific gravity is an analytical value that reflects the porosity of a material. When particle sizes are uniform, the lower the bulk specific gravity, the more porous and the greater the specific surface area. This increases the contact area between an enzyme and a substrate at the time of an enzymatic reaction, thereby promoting the enzymatic reaction.

However, when the particle sizes of the enzyme-immobilization carrier differ, for example, even when materials have the same specific surface area, the larger the particle size, the lower the measured bulk specific gravity.

### Oil Absorption Ratio

The enzyme-immobilization carrier of the present invention is characterized in that it has higher oil absorbability than porous bodies such as textured soybean protein produced by conventional twin-screw extruders.

The enzyme-immobilization carrier of the present invention desirably has high oil absorbability. In a case of immobilizing an enzyme for fats and oils as a substrate, when oil absorbability is high, a large amount of the fats and oils substrate can be brought into contact with the immobilized enzyme accordingly, which makes the progress of the enzymatic reaction efficient.

As an index representing the level of oil absorbability, an oil absorption ratio can be used. In the enzyme-immobilization carrier of the present invention, an oil absorption ratio is 2 times or more by weight and is particularly preferably 3 times or more by weight, but it may be 4 times or more by weight, 5 times or more by weight, or 6 times or more by weight.

On the other hand, in a conventional and commercially available textured soybean protein, an oil absorption ratio is about 0.8 to 1.7 times by weight, meaning that the oil absorbability is not so high.

That is, the enzyme-immobilization carrier of the present invention can exhibit an oil absorption ratio that is three times or more that of conventional textured soybean protein. The oil absorption ratio is measured by the following method.

### Conditions for Measuring Oil Absorption Ratio

100 g of palm oil at 80° C. is added to 10 g of a sample. After absorbing the oil for 20 minutes, the oil is removed with a 30-mesh sieve to measure a weight (Xg) of the sample after absorbing the oil. Then, an oil absorption ratio (Z) is obtained from the following formula.

$$\text{Z =} \left(\text{X} - 10\right) / 10$$

### Water Absorption Ratio

The enzyme-immobilization carrier of the present invention preferably has higher water absorbability than porous bodies such as textured soybean protein produced by conventional twin-screw extruders.

The high water absorbability means that the enzyme-immobilization carrier can retain and adhere a large amount of an aqueous enzyme solution accordingly, and as a result, a large amount of enzymes can be carried. As an index representing the level of water absorbability, a water absorption ratio can be used. In the enzyme-immobilization carrier the present invention, a water absorption ratio is 7 times or more by weight, and is particularly preferably 8 times or more by weight, and preferably 9 times or more by weight.

On the other hand, in a conventional and commercially available textured soybean protein, a water absorption ratio is about 3.3 to 7.4 times by weight. The water absorption ratio is measured by the following method.

### Conditions for Measuring Water Absorption Ratio)

100 g of water at 80° C. is added to 10 g of a sample. After absorbing the water for 20 minutes, the water is removed with a 30-mesh sieve to measure a weight (Xg) of the sample after absorbing the water. Then, a water absorption ratio (Y) is obtained from the following formula.

$$\text{Y =} \left(\text{X} - 10\right) / 10$$

### Form

The enzyme-immobilization carrier of the present invention is typically a granular porous body. In the present invention, the term “granule” means a grain having a particle diameter larger than that of a powder.

Although the size of granules is not particularly limited, it is suitable that 90% by weight or more of the total weight of granules remains on a 42 mesh sieve conforming to the international standard “ISO 3301-1.”

However, the enzyme-immobilization carrier of the present invention can also be used by being appropriately ground, and is, in this case, in the form of finer granules or a powder. In addition, the carrier can also be used by binding each granule together, and may be, in this case, in the form of a mass larger than a granule.

### Textured Protein Material

The enzyme-immobilization carrier of the present invention is typically a granule in a so-called indeterminate form not having a specific and fixed shape, probably because powders aggregate and are bound to each other to form coarse particles by pressurized heating treatment subjected to a raw material powder.

On the other hand, as a granule in a determinate form, there are textured protein materials produced by a twin-screw extruder, extrusion-granulated granules, and the like. The textured protein material is obtained by while swelling a dough, which is formed by kneading raw materials and water, in a device by subjecting the dough to pressurized heating treatment, the dough is extruded under normal pressure from a die in a determinate form attached to the tip end of the device so that in the outlet thereof, the dough is cut to be formed at regular intervals in a determinate form manner. Therefore, the enzyme-immobilization carrier of the present invention is distinguished in shape from a porous body produced by a twin-screw extruder.

### Production of Enzyme-immobilization Carrier

Hereinafter, an embodiment for producing the enzyme-immobilization carrier of the present invention will be specifically described.

### Powdered Plant Protein Material

In order to obtain the enzyme-immobilization carrier in which the protein content is 80% by weight or more per dry weight as in the present invention, it is required to use a raw material with a higher protein content, and a “powdered plant protein material” can be typically used as a raw material. The powdered plant protein material refers to a protein material obtained by powdering a substance in which a part or all of non-protein components such as lipids, soluble carbohydrates, starch, insoluble fibers, and minerals have been removed from a plant raw material to further concentrate the protein content. It is preferable to use a substance in which the protein content is 50% by weight or more in the solid content, and it is more preferable to use a substance in which the protein content is 60% by weight or more, 70% by weight or more, particularly 75% by weight or more, 80% by weight or more, or 90% by weight or more in the solid content.

### Powdered Plant Protein Material

For the powdered plant protein material, a single type may be used, but a plurality of types of powders may be mixed at a desired ratio so as to be provided as a raw material for the production of the carrier. In this case, the above-mentioned protein content in the solid content of the powdered plant protein material means a value of the mixture. In addition, the powdered plant protein material, and if necessary, a powdered animal protein material can be used, for example. More specifically, a powdered soybean protein material and a powdered milk protein material can be provided as a raw material by being mixed at a ratio of 1:10 to 10:1.

In addition, other edible materials other than the powdered plant protein material, or non-edible materials can be appropriately mixed. These are preferably powders, but may be mixed in a liquid state as long as there is no influence on the operation of powder pressurization and heating.

### Production Example of Powdered Plant Protein Material

Herein, with soybeans as an example, a typical and non-limiting production example of a powdered soybean protein material will be described below. Even when other plant raw materials are used, the powdered plant protein material can be produced according to the following production example.

### I) Extraction Step

Water is added to defatted soybean used as a soybean raw material, the mixture is subjected to stirring and the like to form a suspension (slurry), and protein is extracted with water. The water can have a neutral to alkaline pH and can also contain salts such as calcium chloride. The okara (tofu dregs) is separated by solid-liquid separation means such as centrifugation to obtain a protein extraction liquid (so-called soy milk). A powder obtained by heat sterilization and spray drying at this stage is a so-called defatted soy milk powder, which can be used as the powdered plant protein material.

### II) Acid Precipitation Step

Subsequently, an acid such as hydrochloric acid or citric acid is added to the protein extraction liquid to adjust the pH of the extraction liquid to pH 4 to 5, which is the isoelectric point of soybean protein, thereby insolubilizing the protein to cause acid precipitation. Subsequently, a supernatant (so-called whey) containing carbohydrates and ash, which are acid-soluble components, is removed by solid-liquid separation means such as centrifugation to recover an “acid-precipitated curd” containing acid-insoluble components. A powder obtained by spray drying at this stage is a so-called curd powder, which can be used as the powdered plant protein material.

### III) Neutralization Step

Subsequently, water is added to the acid-precipitated curd again, and as necessary, the curd is washed with water to obtain a “curd slurry” thereafter. Then, the slurry is neutralized by adding an alkali such as sodium hydroxide or potassium hydroxide to obtain a “neutralized slurry.”

### IV) Sterilization and Powdering Step

Subsequently, the neutralized slurry is sterilized by heating, spray-dried with a spray dryer or the like, and optionally subjected to fluidized bed granulation to obtain a so-called isolated soybean protein.

However, the powdered soybean protein material is not limited to the one produced in the above-mentioned production example. As the soybean raw material, various soybean raw materials such as full-fat soybean and partially defatted soybean can be used instead of defatted soybean. Also for extraction means, various extraction conditions and devices can be applied. As a method for removing whey from a protein extraction liquid, instead of performing acid precipitation, membrane concentration using an ultrafiltration membrane or the like can be performed, and in this case, the neutralization step is not necessarily required. In addition, production can also be performed by applying a method of extracting protein with neutral or alkaline water after preliminarily washing with acid water or alcohol to remove whey from a soybean raw material. Alternatively, protein can also be partially hydrolyzed by causing a protease to act on a protein solution at any of the above-mentioned steps.

### Solubility of Powdered Plant Protein Material in Water

A solubility itself of the powdered plant protein material obtained as described above in water is generally high, and in the powdered plant protein material, a Nitrogen Solubility Index (NSI) is 60 or more, and is sometimes 65 or more, 70 or more, 75 or more, 80 or more, 82 or more, 85 or more, 90 or more, 92 or more, 94 or more, or 96 or more. When an attempt is made to use the powdered plant protein material itself having these relatively high NSI’s as the enzyme-immobilization carrier, the powder surface dissolves when adhering an aqueous enzyme solution, resulting in encompassing of an enzyme by a protein material, and thereby the enzymatic activity cannot be expressed, which makes the powdered plant protein material not suitable as the immobilization carrier.

### Granulation by Pressurized Heating Treatment in Powdered State

As at least one processing method for obtaining the enzyme-immobilization carrier of the present invention, production can be performed under various conditions in which the above-mentioned powdered plant protein material as a raw material is subjected to pressurized heating treatment in a powdered state instead of an aqueous system by a direct heating method using steam so that the NSI is reduced below that before the pressurized heating treatment. By such pressurized heating treatment, the enzyme-immobilization carrier of the present invention is granulated and at the same time becomes porous, that is, a large surface area structure. In addition, when the NSI is 70 or less, a physical property that makes dissolving in water difficult is obtained, and thereby an enzyme is easily distributed on the surface when adhering an aqueous enzyme solution, which makes it possible to obtain a suitable enzymatic activity.

### Heating Pressure for Pressurized Heating Treatment

The pressure in the pressurized heating treatment can be appropriately set so that the enzyme-immobilization carrier has a desired quality, but preferably, the heating pressure may be 0.03 MPa or more, 0.05 MPa or more, 0.1 MPa or more, 0.2 MPa or more, 0.3 MPa or more, or 0.4 MPa or more, and the heating pressure may be 0.7 MPa or less, 0.6 MPa or less, 0.5 MPa or less, or 0.4 MPa or less. Furthermore, a range of 0.05 MPa to 0.7 MPa may be selected as one preferable embodiment.

### Temperature in Pressurized Heating Treatment

The temperature in the pressurized heating treatment varies depending on the pressure and is a temperature exceeding 100° C. because of the pressurized state, but depending on embodiments, the temperature may be 120° C. or higher, 130° C. or higher, 140° C. or higher, 150° C. or higher, 160° C. or higher, or 170° C. or higher. Although the upper limit of the temperature is not set, the temperature is usually 250° C. or lower.

### Heating Time for Pressurized Heating Treatment

The heating time of the pressurized heating treatment can be appropriately set in consideration of the combination with the heating temperature so that the enzyme-immobilization carrier has a desired quality, but a short time is preferable, and the heating time may be 1 minute or shorter, 30 seconds or shorter, 20 seconds or shorter, 10 seconds or shorter, 5 seconds or shorter, 2 seconds or shorter, 1 second or shorter, and particularly 0.5 seconds or shorter, or 0.3 seconds or shorter. In addition, the heating time may be 0.00001 seconds or longer, 0001 seconds or longer, or 0.001 seconds or longer. Furthermore, as one preferable embodiment, a range of 0.00001 to 2 seconds, 0.0001 to 1 second, or 0.001 to 0.5 seconds may be selected.

### Direct Heating Method

Heating methods for the pressurized heating treatment are broadly classified into a direct heating method and an indirect heating method. A preferable embodiment for obtaining the enzyme-immobilization carrier of the present invention adopts a direct heating method using steam. Examples of powder heat treatment apparatuses capable of performing such pressurized heating treatment include “KPU” (OKAWARA MFG. CO., LTD.), “SKS-50” (SEISHIN ENTERPRISE Co., Ltd.), “Sonic Stera” (manufactured by Fujiwara Techno-Art Co., Ltd.), which are airflow type powder sterilizers, and improved types of them. As described above, by the direct heating method using steam such as superheated steam, the powder of the powdered plant protein material is directly exposed to steam and subjected to the pressurized heating treatment, and thereby the powdered plant protein material can be aggregated and granulated.

### Vertical Type

Furthermore, in the present invention, it is preferable to perform the pressurized heating treatment by the direct heating method using steam while dropping the powdered plant protein material in a powdered state in a vertically direction in the pressurized heating treatment by the direct heating method. A heating and pressurizing device for implementing such a heating method is preferably a device having a heating space of a closed system in which a powder introduced into the device can be dropped in a vertical direction, and having a mechanism for bringing the powder into contact with steam in a pressurized state during dropping of the powder in the space. In the present invention, such a pressurized heating device is called a “vertical type.” As an aspect of the vertical type, a sterilizer for powders and granules as disclosed in Patent Literature 6 can be applied as the pressurized heating device, and specifically, commercially available “Sonic Stera” (manufactured by Fujiwara Techno-Art Co., Ltd.) can be used.

Accordingly, the NSI of the powdered plant protein material can be efficiently reduced to a desired range, making it possible to produce the enzyme-immobilization carrier that has higher water absorbability and is suitable for immobilizing enzymes.

### Horizontal Type

On the other hand, when the powder is heated using a highly water-soluble plant protein material as a raw material and using a pressurized heating device of a so-called “horizontal type” in which a heating space of a closed system that is pressurized and heated by steam is disposed in a horizontal direction, the powder may stick to the inside of the device, making the efficiency of the production inefficient. In addition, the mechanism of the pressurized heating device of the horizontal type is unclear, probably because it is difficult for the horizontal type to perform pressurized heating in an extremely short time unlike the vertical type, but according to Patent Literature 7, it is disclosed that the water absorption ratio of the obtained granules is about 2 to 3 times by weight, indicating that water absorbability is not sufficient.

### Conventional Textured Protein Material

In addition, a twin-screw extruder, which has been used for the production of conventional textured protein materials, is also used as a powder sterilizer. However, the twin-screw extruder is for pressurized heating treatment of an indirect heating method and is not for a heating method in which a powder is directly exposed to steam, and thus is for a method that is completely different from the pressurized heating treatment of the present invention.

### Grinding, Crushing, Classification, and Binding

The granules produced as described above can be directly used as a product as the enzyme-immobilization carrier. In addition, if necessary, the granules can be further processed, and can be ground or crushed to a suitable particle size, for example. Alternatively, it is possible to obtain an enzyme-immobilization carrier in which particle size regulation has been performed by providing the granules to a classifier to fractionate them into granules having a desired particle size range. Alternatively, the granules can also be bound together to be formed into lumps of a certain size.

### Method for Producing Immobilized Enzyme

A method for producing an immobilized enzyme of the present invention is for adhering an aqueous solution containing an enzyme to an enzyme-immobilization carrier and thereafter drying. For adhesion, the carrier may be immersed in the aqueous enzyme solution, or this solution may be sprayed onto the carrier. When the enzyme is powder, it is used after being dissolved in water, and when the enzyme is liquid, it is used after being diluted as necessary.

When the enzyme is lipase, a transesterification activity is improved when a small amount of fats and oils is added to the aqueous enzyme solution and dispersed before adhering the aqueous enzyme solution to the carrier.

The transesterification reaction of fats and oils can be caused by using the immobilized enzyme of the present invention. Because transesterified fats and oils are mainly used as food, when the immobilization carrier is food, it can be reliably used.

### EXAMPLES

The embodiments of the present invention will be described in more detail below with reference to examples. Note that “%” and “parts” in the examples indicate “% by weight” and “parts by weight” unless otherwise specified.

### Pressurized Heating-Treated Product A

The powdered plant protein material in a powdered state was subjected to pressurized heating treatment of a direct heating method using steam to produce an enzyme-immobilization carrier.

As a sample of the powdered plant protein material, a commercially available powdered soybean protein “Fujipro F” (manufactured by FUJI OIL CO., LTD.) was used. The present sample had a protein content of 91.2% and an NSI of 98.6, and was a highly water-soluble type.

As a pressurized heating device, a commercially available “Sonic Stera” (manufactured by Fujiwara Techno-Art Co., Ltd.) was used. The present device is a vertical type device capable of pressurized heating treatment by a direct heating method using steam while dropping a powder in a vertical direction in a heating space.

A treated product, which was obtained by the pressurized heating treatment with a heating pressure of 0.2 MPa and a heating time of 0.2 seconds, was passed through a 20 M (850 µm) sieve, and the fraction that remained on a 100 M (150 µm) sieve was classified to obtain a pressurized heating-treated product A. The pressurized heating-treated product A had a bulk specific gravity of 0.26 g/cm³, an NSI of 64.8, a water supply ratio of 7.5, and an oil absorption ratio of 2.5 (which are shown in Table 1).

### Pressurized Heating-Treated Products B to D

The pressurized heating treatment was performed at a heating pressure of 0.6 MPa and a heating time of 0.2 seconds using the same powdered plant protein material and pressurized heating device as in the pressurized heating-treated product A. This treated product was passed through a 20 M (850 µm) sieve, and the fraction that remained on a 100 M (150 µm) sieve was classified to obtain a pressurized heating-treated product B.

Furthermore, the treated product was passed through a 3.5 M (5.6 mm) sieve, and the fraction that remained on a 20 M (850 µm) sieve was classified to obtain a pressurized heating-treated product C having a relatively large particle size.

Furthermore, the fraction that had passed through a 100 M (150 mm) sieve was classified to obtain a pressurized heating-treated product D having a relatively small particle size.

Table 1 shows the bulk specific gravity, the NSI, the water supply ratio, and the oil absorption ratio of the pressurized heating-treated products B to D thus obtained.

### Examples 1 to 4

Immobilized enzymes of Examples 1 to 4 were respectively prepared according to the formulations shown in Table 2 using the pressurized heating-treated products A to D as enzyme-immobilization carriers. A preparation method was performed according to a “◯ Method for preparing immobilized enzyme.” Using the obtained immobilized enzymes, the transesterification activity was evaluated. A method was performed according to a “◯ Method for evaluating immobilized enzyme.” Table 2 shows the results.

### Comparative Example 1

A transesterification activity was evaluated after preparing an immobilized enzyme of Comparative Example 1 according to the formulation shown in Table 2 using diatomaceous earth (trade name: Celite No. 545, manufactured by FUJIFILM Corporation) as an enzyme-immobilization carrier. Table 2 shows the results. Diatomaceous earth has been conventionally used as a carrier that immobilizes enzymes, but resources are being depleted in recent years, and substitutes therefor have been desired.

### Method for Preparing Immobilized Enzyme

According to the formulation of the immobilized enzyme formulation in Table 2, the following procedure was performed.

1. A powdered enzyme preparation (lipase derived from microorganisms) and water were mixed.

2. After adding a high oleic sunflower oil (trade name: Haioru 75B, manufactured by FUJI OIL CO., LTD.) as fats and oils to 1, the mixture was stirred to prepare an enzyme solution. The emulsified state was O/W type.

3. 2 was added to the enzyme-immobilization carrier and mixed uniformly with a spatula.

4. Drying was performed in a vacuum environment until the moisture content was 1% or less.

### Method for Evaluating Immobilized Enzyme

1. A palm olein (IV56) was dehydrated, and by confirming that the water amount was 180 to 220 ppm, a fats and oils substrate was prepared.

2. In an Erlenmeyer flask, 50 g of the fats and oils substrate was added, and the immobilized enzyme was added so that the amount of the enzyme preparation added with respect to the substrate was 0.008% of the fats and oils substrate.

3. The mixture was shaken at 60° C. and 200 rpm, and after reacting for 24 hours, sampling was performed.

4. A triglyceride (TG) composition was analyzed by gas chromatography (GC).

(The present evaluation method is an evaluation method based on a transesterification reaction, and it could be determined that the reaction progressed as the amount of a target product (C48) increased with respect to main components (C48 to C56) of the fats and oils substrate.)

5. A transesterification rate (%) was calculated from the TG composition.
Transesterification rate (%) = (C48 - C48 (0 time))/(C48 (END) - C48 (0 time)) Herein,
C48 = peak area of C48 by GC/(sum of peak areas of C48 to C56 by GC) × 100,
C48 (0 time) indicates C48 of the fats and oils substrate when it was unreacted, and
C48 (END) indicates C48 when the transesterification reaction was completed.

6. With a transesterification rate of Comparative Example 1 using diatomaceous earth that is a conventional product as 100, when a relative value thereof was 75 or more, this was determined to be acceptable.

### Evaluation Results

In all of the immobilized enzymes of Examples 1 to 4 using the enzyme-immobilization carrier that was a porous body containing plant protein and had a protein content of 80% by weight or more, an NSI of 70 or less, and an oil absorption ratio of 2 times or more, excellent results in which the reaction rate relative value exceeded 100 were obtained, indicating that all of them were acceptable. In particular, a very excellent result of 135 was obtained for Example 4.

### Comparative Example 2

A transesterification activity was evaluated after preparing an immobilized enzyme of Comparative Example 2 according to the formulation shown in Table 2 and in the same manner as in Example 1 except that a commercially available isolated soybean protein “Fujipro F” (manufactured by FUJI OIL CO., LTD.), which is a powdered soybean protein material, was used as the enzyme-immobilization carrier. The reaction rate relative value was 16, which was very poor. As shown in Table 1, “Fujipro F” had a high NSI of 98.6.

### Comparative Example 3

A transesterification activity was evaluated after preparing an immobilized enzyme of Comparative Example 3 according to the formulation shown in Table 2 and in the same manner as in Example 1 except that a commercially available textured soybean protein “Apex 650” (manufactured by FUJI OIL CO., LTD.), which is a textured soybean protein material, was used as the enzyme-immobilization carrier. The reaction rate relative value was 9, which was very poor. As shown in Table 1, “Apex 650” had a low protein content of 74.8% and a low oil absorption ratio of 1.7.

### Comparative Example 4

A transesterification activity was evaluated after preparing an immobilized enzyme of Comparative Example 4 according to the formulation shown in Table 2 and in the same manner as in Example 1 except that a product, which was obtained by finely grinding a commercially available textured soybean protein “Apex 650” (manufactured by FUJI OIL CO., LTD.), which is a textured soybean protein material, using a grinding device “SCM-50” (manufactured by SHIBATA) so that the average particle diameter was 60 to 70 µm, was used as the enzyme-immobilization carrier. The reaction rate relative value was 10, which was very poor. As shown in Table 1, the finely ground product of “Apex 650” had a low protein content of 74.8% and a low oil absorption ratio of 0.8.

### Comparative Example 5

A transesterification activity was evaluated after preparing an immobilized enzyme of Comparative Example 5 according to the formulation shown in Table 2 and in the same manner as in Example 1 except that a commercially available microcrystalline cellulose “ST-100” (manufactured by Asahi Kasei Corporation), which is a plant material, was used as the enzyme-immobilization carrier. The reaction rate relative value was 46, which was poor.

### Summary

From the above results, it was found that in the immobilized enzymes of Examples 1 to 4 using the enzyme-immobilization carrier that was a porous body containing plant protein and had a protein content of 80% by weight or more, an NSI of 70 or less, and an oil absorption ratio of 2 times or more by weight, the same or higher level of the transesterification activity could be expressed as compared to diatomaceous earth for which resources are being depleted in recent years and substitutes have been desired. In addition, because people have experience eating the powdered plant protein material, which is the raw material of the enzyme-immobilization carrier of the present invention, there is also the benefit of feeling relief when applied to food materials.

TABLE 1

| Immobilized enzyme | Immobilization carrier | Protein content (%) | Pressuriz ed heating treatment | Heating pressure (MPa) | Heatin g time (sec) | Bulk specifi c gravity (g/cm³) | NSI | Water absorptio n ratio | Oil absorptio n ratio |
|---|---|---|---|---|---|---|---|---|---|
| Example 1 | Pressurized | 91.2 | Performed | 0.2 | 0.2 | 0.26 | 64.8 | 7.5 | 2.5 |
| | heating-treated product A | | | | | | | | |
| Example 2 | Pressurized heating-treated product B | 91.2 | Performed | 0.6 | 0.2 | 0.10 | 20.2 | 9.8 | 6.5 |
| Example 3 | Pressurized heating-treated product C | 91.2 | Performed | 0.6 | 0.2 | 0.08 | 21.0 | 10.3 | 6.4 |
| Example 4 | Pressurized heating-treated product D | 91.2 | Performed | 0.6 | 0.2 | 0.23 | 22.0 | 9.2 | 6.4 |
| Comparative Example 1 | Diatomaceous earth | - | - | - | - | - | - | - | - |
| Comparative Example 2 | Powdered soybean protein | 91.2 | Untreated | - | - | 0.30 | 98.6 | - | - |
| Comparative Example 3 | Textured soybean protein | 74.8 | Twin-screw extruder | | | 0.18 | 11.0 | 7.4 | 1.7 |
| Comparative Example 4 | Textured soybean protein-ground product | 74.8 | Twin-screw extruder | | | 0.35 | 11.0 | 4.2 | 0.8 |
| Comparative Example 5 | Microcrystallin e cellulose | - | - | - | - | 0.12 | - | - | - |

TABLE 2

| Immobilized enzyme | Enzyme-immobiliza tion carrier | Immobilized enzyme preparation formulation (parts) | | | | Evaluation result of transesterification activity | | | | | | |
|---|---|---|---|---|---|---|---|---|---|---|---|---|
| | | Carrie r | Water | Enzyme preparatio n | Fats and oils | Enzyme preparation in immobilized enzyme (%) | Amount of immobilize d enzyme added with respect to substrate (%) | Amount of enzyme preparation added with respect to substrate (%) | AV of reaction oil | Reaction rate X (%) | | Relative value of reaction rate with Comparative Example 1 as 100 |
| Example 1 | Pressurized heating-treated product A | 100.0 | 96.0 | 4.0 | 12.0 | 3.42 | 0.234 | 0.008 | 1.5 | | 42.0 | 102 |
| Example 2 | Pressurized heating-treated product B | 100.0 | 96.0 | 4.0 | 12.0 | 3.42 | 0.234 | 0.008 | 1.6 | | 47.9 | 116 |
| Example 3 | Pressurized heating-treated product C | 100.0 | 96.0 | 4.0 | 12.0 | 3.42 | 0.234 | 0.008 | 1.7 | | 42.0 | 102 |
| Example 4 | Pressurized heating-treated product D | 100.0 | #### | 4.8 | 14.4 | 4.00 | 0.200 | 0.008 | 1.6 | | 55.5 | 135 |
| Comparative Example 1 | Diatomaceous earth | 100.0 | 96.0 | 4.0 | 12.0 | 3.42 | 0.234 | 0.008 | 1.6 | | 41.2 | 100 |
| Comparative Example 2 | Powdered soybean protein | 100.0 | 41.2 | 1.7 | 5.1 | 1.59 | 0.503 | 0.008 | 1.6 | | 6.4 | 16 |
| Comparative Example 3 | Textured soybean protein | 100.0 | 96.0 | 4.0 | 12.0 | 3.42 | 0.234 | 0.008 | 1.7 | | 3.7 | 9 |
| Comparative Example 4 | Textured soybean protein-ground product | 100.0 | 96.0 | 4.0 | 12.0 | 3.42 | 0.234 | 0.008 | 1.7 | | 4.3 | 10 |
| Comparative Example 5 | Microcrystalline cellulose | 100.0 | 96.0 | 4.0 | 12.0 | 3.42 | 0.234 | 0.008 | 1.7 | | 18.9 | 46 |

### INDUSTRIAL APPLICABILITY

The enzyme-immobilization carrier of the present invention which is based on plant protein can be used as a novel and reproducible plant-based enzyme-immobilization carrier that is biodegradable, thereby imposing a low environmental load, and is also harmless for a human body. Therefore, utilization of the enzyme-immobilization carrier of the present invention is expected in enzymatic reaction processes, particularly in the fields of foods and pharmaceuticals.

1. An enzyme-immobilization carrier which has all of the following characteristics A to D:
A, the enzyme-immobilization carrier is a porous body including plant protein,
B, a content of the protein is 80% by weight or more per dry weight,
C, a nitrogen solubility index (NSI) is 70 or less, and
D, an oil absorption ratio is 2 times or more by weight.

2. The enzyme-immobilization carrier according to claim 1,
wherein the enzyme-immobilization carrier further has both of the following characteristics E and F:
E, a water absorption ratio is 7 times or more by weight, and
F, a bulk specific gravity is 0.28 g/cm³ or less.

3. An immobilized enzyme, wherein the enzyme is immobilized on the enzyme-immobilization carrier according to claim 1.

4. The immobilized enzyme according to claim 3, wherein the enzyme is for fats and oils as a substrate.

5. A method for producing an immobilized enzyme, the method comprising:
adhering an aqueous solution containing an enzyme for fats and oils as a substrate to an enzyme-immobilization carrier having all of the following characteristics A to D; and
drying thereafter,
A, the enzyme-immobilization carrier is a porous body including plant protein,
B, a content of the protein is 80% by weight or more per dry weight,
C, a nitrogen solubility index (NSI) is 70 or less, and
D, an oil absorption ratio is 2 times or more by weight.

6. A method for immobilizing an enzyme, the method comprising:
adhering an aqueous solution containing an enzyme for fats and oils as a substrate to an enzyme-immobilization carrier having all of the following characteristics A to D; and
drying thereafter,
A, the enzyme-immobilization carrier is a porous body including plant protein,
B, a content of the protein is 80% by weight or more per dry weight,
C, a nitrogen solubility index (NSI) is 70 or less, and
D, an oil absorption ratio is 2 times or more by weight.

7. A method for producing transesterified fats or oils, the method comprising
performing a transesterification reaction using an immobilized enzyme obtained by adhering an aqueous solution containing an enzyme for fats and oils as a substrate to an enzyme-immobilization carrier having all of the following characteristics A to D, and drying thereafter,
A, the enzyme-immobilization carrier is a porous body including plant protein,
B, a content of the protein is 80% by weight or more per dry weight,
C, a nitrogen solubility index (NSI) is 70 or less, and
D, an oil absorption ratio is 2 times or more by weight.

8. An immobilized enzyme, wherein the enzyme is immobilized on the enzyme-immobilization carrier according to claim 2.
